(19) United States
(12) Patent Application Publication
(10) Pub. No.: US 2020/0333338 A1
(43) Pub. Date: Oct. 22, 2020

(54) PATHOGEN DETECTION APPARATUS AND PATHOGEN DETECTION METHOD

(71) Applicant: Panasonic Intellectual Property Management Co., Ltd., Osaka (JP)

(72) Inventors: SHUNSUKE KORI, Osaka (JP); KAZUAKI NISHIO, Osaka (JP)

(21) Appl. No.: 16/917,991

(22) Filed: Jul. 1, 2020

Related U.S. Application Data
(63) Continuation of application No. PCT/JP2019/007432, filed on Feb. 27, 2019.

(30) Foreign Application Priority Data
Apr. 6, 2018 (JP) 2018-073966

(51) Int. Cl. G01N 33/569; G01N 1/22; G01N 33/58; G01N 33/553; G01N 33/543; A61B 5/01

(52) U.S. Cl. CPC G01N 33/569; G01N 1/22; G01N 33/582; G01N 2458/00; G01N 33/54366; A61B 5/01; G01N 2001/2244; G01N 33/553

(57) ABSTRACT

A pathogen detection apparatus includes an obtainer that obtains a body temperature of a subject; a collector that collects a pathogen carried by the subject or a pathogen in air around the subject; a detector that performs detection of the pathogen collected by the collector; a reporter that reports a detection result obtained by the detector; and a controller. In a case where the body temperature of the subject obtained by the obtainer is higher than a predetermined threshold value, the controller controls at least one of the collector or the detector to shorten a time period from start of collection by the collector to report of the detection result by the reporter.

### BACKGROUND

### 1. Technical Field

The present disclosure relates to a pathogen detection apparatus and a pathogen detection method that efficiently detect a virus in accordance with a subject.

### 2. Description of the Related Art

In recent years, the spread of infectious diseases, such as influenza, in nursing homes, hospitals, schools, and the like has been a social issue, and the diffusion of techniques capable of detecting a virus has been expected for purposes besides medical purposes. For example, Japanese Unexamined Patent Application Publication No. 2015-178993 proposes a technique capable of detecting a virus. In this technique, pathogens such as viruses suspended in the air are collected, the concentration of a pathogen such as a virus contained in the air is measured by using fluorescence spectroscopy, surface-enhanced Raman scattering spectroscopy, an immunochromatographic device using an antigen-antibody reaction, or the like, and thereby the concentration of the virus suspended in the air is measured.

The following literature discloses that the quantity of virus carried by an infected person is correlated with his/her body temperature and that the body temperature is proportional to the quantity of virus: Lincoln L. H. Lau, Benjamin J. Cowling, Vicky J. Fang, Kwok-Hung Chan, Eric H. Y. Lau, Marc Lipsitch, Calvin K. Y. Cheng, Peter M. Houck, Timothy M. Uyeki, J. S. Malik Peiris, and Gabriel M. Leung, “Viral Shedding and Clinical Illness in Naturally Acquired Influenza Virus Infections”, The Journal of Infectious Diseases, 201 1509-1516 (2010).

### SUMMARY

In the virus detection techniques according to the related art, similar measurement methods are always used regardless of the conditions of subjects, and thus similar time periods are taken to determine whether the subjects are infected or not infected.

One non-limiting and exemplary embodiment provides a pathogen detection apparatus and a pathogen detection method that are capable of efficiently detecting a pathogen from a subject or a space around the subject.

In one general aspect, the techniques disclosed here feature a pathogen detection apparatus including an obtainer that obtains a body temperature of a subject; a collector that collects a pathogen carried by the subject or a pathogen in air around the subject; a detector that performs detection of the pathogen collected by the collector; a reporter that reports a detection result obtained by the detector; and a controller. In a case where the body temperature of the subject obtained by the obtainer is higher than a predetermined threshold value, the controller controls at least one of the collector or the detector to shorten a time period from start of collection by the collector to report of the detection result by the reporter.

It should be noted that general or specific embodiments may be implemented as a method, a system, an integrated circuit, a computer program, a computer-readable recording medium, or any selective combination thereof. The computer-readable recording medium includes, for example, a nonvolatile recording medium, such as a compact disc-read only memory (CD-ROM).

According to one embodiment of the present disclosure, a virus can be efficiently detected from a subject or a space around the subject. Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a diagram illustrating an example of the external appearance of a pathogen detection apparatus according to an embodiment;

FIG. 2 is a schematic configuration diagram of the pathogen detection apparatus according to the embodiment;

FIG. 3 is a diagram for describing the function of a cyclone according to the embodiment;

FIG. 4 is a configuration diagram of a detection device according to the embodiment;

FIG. 5 is a diagram for describing the details of an antigen-antibody reaction;

FIG. 6 is a diagram illustrating an example of a substrate structure in the case of using surface plasmon resonance;

FIG. 7 is a block diagram illustrating an example of the functional configuration of the pathogen detection apparatus according to the embodiment;

FIG. 8 is a diagram including a graph showing the relationship between a reaction time period and a detection signal in different virus concentrations;

FIG. 9 is a diagram including a graph showing the relationship between the quantity of virus carried by a subject and a body temperature of the subject;

FIG. 10 is a flowchart illustrating an example of a pathogen detection method for the pathogen detection apparatus according to the embodiment;

FIG. 11 is a diagram for describing a first control mode and a second control mode in the pathogen detection apparatus according to the embodiment;

FIG. 12 is a diagram for describing a first detection mode and a second detection mode in the pathogen detection apparatus according to the embodiment;

FIG. 13 is a diagram for describing a first control mode and a second control mode in the pathogen detection apparatus according to a first modification example;

FIG. 14 is a diagram for describing a first collection mode and a second collection mode in the pathogen detection apparatus according to the first modification example;

FIG. 15 is a diagram for describing a first detection mode and a second detection mode in the pathogen detection apparatus according to a second modification example;

FIG. 16 is a flowchart illustrating an example of a pathogen detection method for the pathogen detection apparatus according to a third modification example;

FIG. 17 is a flowchart illustrating an example of a pathogen detection method for the pathogen detection apparatus according to a fourth modification example; and

FIG. 18 is a diagram for describing first to third control modes in the pathogen detection apparatus according to the fourth modification example.

### DETAILED DESCRIPTION

To address the above-described issues, a pathogen detection apparatus according to an aspect of the present disclosure includes an obtainer that obtains a body temperature of a subject; a collector that collects a pathogen carried by the subject or a pathogen in air around the subject; a detector that performs detection of the pathogen collected by the collector; a reporter that reports a detection result obtained by the detector; and a controller. In a case where the body temperature of the subject obtained by the obtainer is higher than a predetermined threshold value, the controller controls at least one of the collector or the detector to shorten a time period from start of collection by the collector to report of the detection result by the reporter.

Accordingly, in a case where the body temperature of the subject is higher than the predetermined threshold value, it is determined that there is a high possibility that the subject is infected with the pathogen in concentration higher than a predetermined concentration, and it is determined that the pathogen is likely to be detected from the subject or the space around the subject. Thus, in a case where there is a high possibility that the subject is infected with the pathogen, the pathogen may be detected even if the time period to obtain a detection result is shorter than in a case where there is a low possibility that the subject is infected with the pathogen. Thus, the pathogen can be efficiently detected from the subject or the space around the subject.

In a case where the body temperature of the subject obtained by the obtainer is lower than or equal to the predetermined threshold value, the controller may control the detector in a first detection mode in which the detector detects the pathogen for a first time period. In a case where the body temperature of the subject is higher than the predetermined threshold value, the controller may control the detector in a second detection mode in which the detector detects the pathogen for a second time period shorter than the first time period.

Accordingly, in a case where the body temperature of the subject is higher than the predetermined threshold value, it is determined that there is a high possibility that the subject is infected with the pathogen in concentration higher than a predetermined concentration, and the time period taken for detection is shorter than in a case where the body temperature of the subject is lower than or equal to the predetermined threshold value. In other words, in this case, the pathogen is more likely to be detected even if the time period for detection is shortened than in a case where the body temperature of the subject is lower than or equal to the predetermined threshold value. Thus, the pathogen can be efficiently detected from the subject or the space around the subject.

The detector may include a reactor that causes a reaction to occur between the pathogen collected by the collector and a labeled substance, and a light irradiator that irradiates, with excitation light, a reacted substance obtained through the reaction in the reactor. In the first detection mode, the detector may detect the pathogen on the basis of fluorescence generated by the labeled substance as a result of irradiating, with the excitation light, the reacted substance obtained through the reaction for the first time period. In the second detection mode, the detector may detect the pathogen on the basis of the fluorescence generated by the labeled substance as a result of irradiating, with the excitation light, the reacted substance obtained through the reaction for the second time period shorter than the first time period.

Accordingly, in a case where the body temperature of the subject is higher than the predetermined threshold value, it is determined that there is a high possibility that the subject is infected with the pathogen in concentration higher than a predetermined concentration, and the time period taken for reaction in detection is shorter than in a case where the body temperature of the subject is lower than or equal to the predetermined threshold value. In other words, in this case, the pathogen is more likely to be detected even if the time period for reaction in detection is shortened than in a case where the body temperature of the subject is lower than or equal to the predetermined threshold value. Thus, the pathogen can be efficiently detected from the subject or the space around the subject.

The detector may be capable of performing, on the pathogen collected by the collector, a pretreatment for promoting the detection. In a case where the body temperature of the subject obtained by the obtainer is lower than or equal to the predetermined threshold value, the controller may cause the detector to perform the pretreatment in the detection. In a case where the body temperature of the subject is higher than the predetermined threshold value, the controller may cause the detector to omit the pretreatment in the detection.

Accordingly, in a case where the body temperature of the subject is higher than the predetermined threshold value, it is determined that there is a high possibility that the subject is infected with the pathogen in concentration higher than a predetermined concentration, and the time period taken for detection is shortened by omitting the pretreatment in the detection. In other words, in this case, the pathogen is more likely to be detected even if the pretreatment is omitted than in a case where the body temperature of the subject is lower than or equal to the predetermined threshold value. Thus, the pathogen can be efficiently detected from the subject or the space around the subject.

In a case where the body temperature of the subject obtained by the obtainer is lower than or equal to the predetermined threshold value, the controller may control the collector in a first collection mode in which the collector collects the pathogen for a third time period. In a case where the body temperature of the subject is higher than the predetermined threshold value, the controller may control the collector in a second collection mode in which the collector collects the pathogen for a fourth time period shorter than the third time period.

Accordingly, in a case where the body temperature of the subject is higher than the predetermined threshold value, it is determined that there is a high possibility that the subject is infected with the pathogen in concentration higher than a predetermined concentration, and the time period taken for collection is shorter than in a case where the body temperature of the subject is lower than or equal to the predetermined threshold value. In other words, in this case, the pathogen is more likely to be detected even if the time period for collection is shortened than in a case where the body temperature of the subject is lower than or equal to the predetermined threshold value. Thus, the pathogen can be efficiently detected from the subject or the space around the subject.

It should be noted that general or specific embodiments may be implemented as a system, a method, an integrated circuit, a computer program, a computer-readable recording medium such as a CD-ROM, or any selective combination thereof.

Hereinafter, a pathogen detection apparatus and a pathogen detection method that relate to one aspect of the present disclosure will be described in detail with reference to the drawings.

The embodiment described below is one specific example of the present disclosure. The values, shapes, materials, components, arrangement positions and connection styles of the components, steps, order of steps, and so forth described in the following embodiment are merely examples and do not limit the present disclosure. Among the components described in the following embodiment, a component that is not described in an independent claim stating the broadest concept will be described as an optional component.

### EMBODIMENT

### Overview of Pathogen Detection Apparatus

A pathogen detection apparatus is an apparatus that has a collection function capable of colleting viruses suspended in the air, such as an influenza virus, and a function of detecting a virus by testing an extraction liquid containing the collected viruses. In particular, the detection is performed by using antibodies that bind specifically to virus components contained in the extraction liquid containing the viruses, with use of a function in which antibodies bind specifically to antigens.

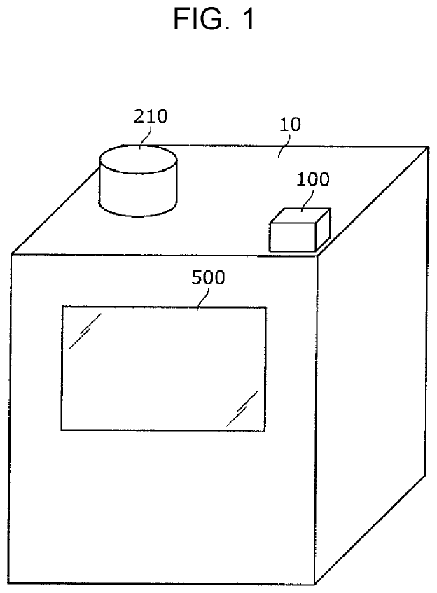

FIG. 1 is a diagram illustrating an example of the external appearance of a pathogen detection apparatus 10 according to an embodiment.

The pathogen detection apparatus 10 is configured to collect, for example, breath exhaled by a subject directly from the subject. The pathogen detection apparatus 10 includes, as illustrated in FIG. 1, for example, a body temperature measurement device 100 that measures a body temperature of a person, an air intake port 210 for collecting air as a detection target, and a display device 500 that displays a detection result, which are exposed on the outer side of a housing. The external appearance of the pathogen detection apparatus 10 illustrated in FIG. 1 is an example, and the configuration is not limited thereto.

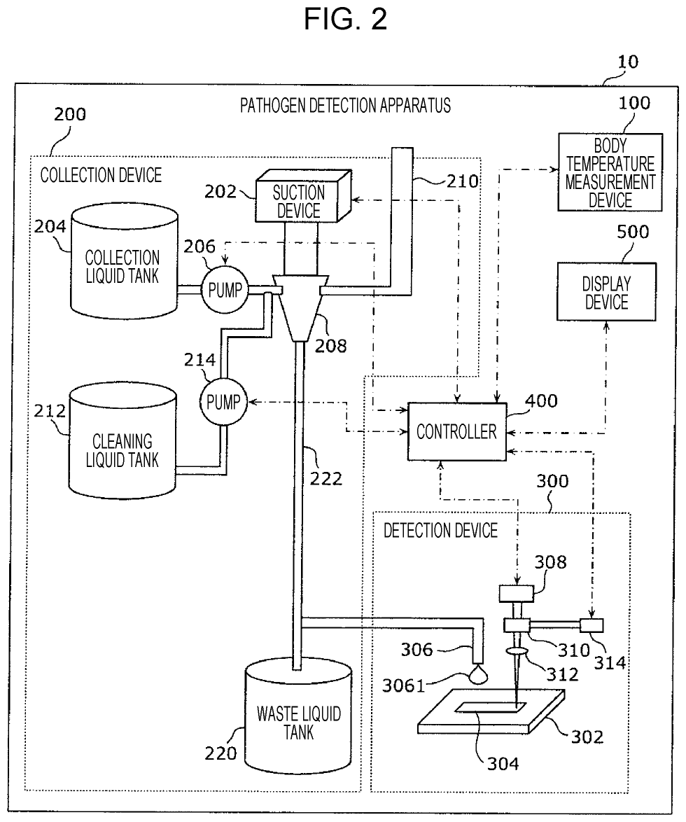

FIG. 2 is a schematic configuration diagram of the pathogen detection apparatus 10 according to the embodiment.

As illustrated in FIG. 2, the pathogen detection apparatus 10 includes the body temperature measurement device 100, a collection device 200, a detection device 300, a controller 400, and the display device 500. Hereinafter, the details of the body temperature measurement device 100, the collection device 200, the detection device 300, the controller 400, and the display device 500 will be described.

Body Temperature Measurement Device

The body temperature measurement device 100 is a temperature sensor that measures a body temperature of a subject. The body temperature measurement device 100 is, for example, a contact temperature sensor that measures a body temperature of a subject by touching the body of the subject or a non-contact temperature sensor that measures a body temperature of a subject without touching the body of the subject. The contact temperature sensor is, for example, a temperature sensor using a thermocouple or the like. The non-contact temperature sensor is, for example, a temperature sensor that measures a body temperature by measuring the quantity of infrared emitted by the body of a subject by using an infrared sensor. The body temperature measurement device 100 is not limited to the above-described examples, and another device according to the related art may be used as long as a body temperature of a subject can be measured. The body temperature measurement device 100 may include a memory that stores a body temperature measurement result. In addition, the body temperature measurement device 100 may store in the memory, as a body temperature measurement result, the date and time of measurement of a body temperature or identification information for identifying a subject having the body temperature in association with the measured body temperature. The body temperature measurement device 100 outputs the body temperature measurement result to the controller 400.

### Configuration of Collection Device

The collection device 200 collects microparticles that may contain viruses in the air and mixes the microparticles into a collection liquid. As illustrated in FIG. 2, the collection device 200 includes a suction device 202, a collection liquid tank 204, a pump 206, a cyclone 208, an air intake port 210, a cleaning liquid tank 212, a pump 214, a waste liquid tank 220, and a liquid channel 222. Hereinafter, the individual components of the collection device 200 will be described.

The suction device 202 sucks in the surrounding atmospheric air through the air intake port 210. Microparticles that may contain viruses suspended in the surrounding atmospheric air are sucked into the cyclone 208 through the air intake port 210 together with the air.

The collection liquid tank 204 is a container for holding a collection liquid for collecting viruses in the air.

The pump 206 supplies the cyclone 208 with the collection liquid in the collection liquid tank 204.

The cyclone 208 is connected to the air intake port 210 and the collection liquid tank 204, and mixes the microparticles that may contain viruses in the air sucked by the suction device 202 through the air intake port 210 and the collection liquid supplied from the collection liquid tank 204 by the pump 206. The cyclone 208 is connected to the detection device 300 via the liquid channel 222. The collection liquid mixed with the microparticles (hereinafter referred to as a specimen) is discharged from the cyclone 208 to the detection device 300 via the liquid channel 222.

The cleaning liquid tank 212 is a container for holding a cleaning liquid for cleaning the cyclone 208 and the liquid channel 222. The cleaning liquid tank 212 is connected to the cyclone 208, and the cleaning liquid in the cleaning liquid tank 212 is supplied to the cyclone 208 by the pump 214.

The waste liquid tank 220 is a container for storing an unnecessary liquid.

The liquid channel 222 is a path for leading a specimen output from the cyclone 208 to the detection device 300.

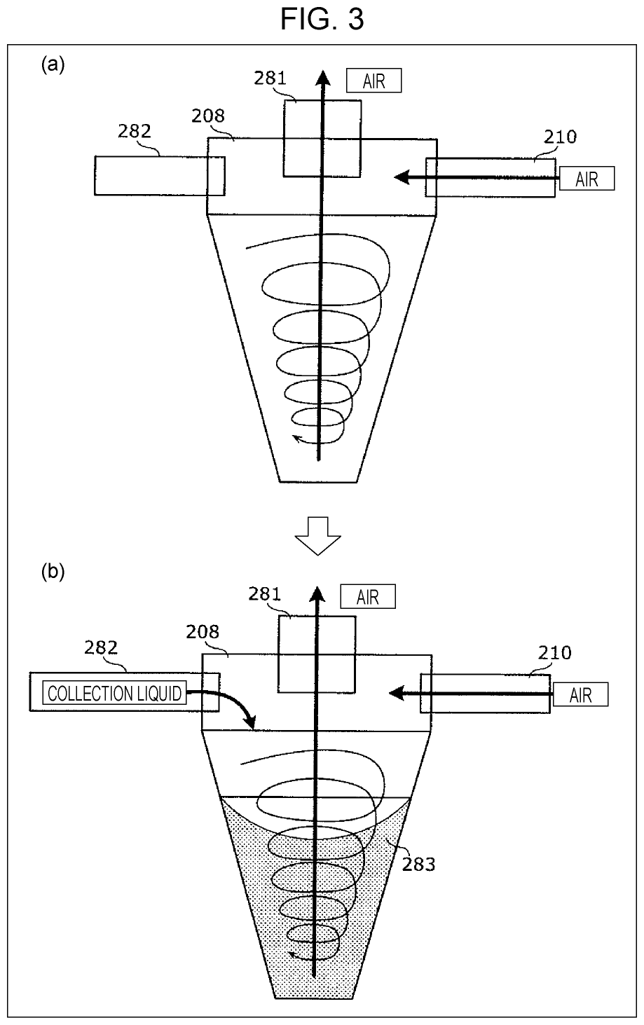

FIG. 3 is a diagram for describing the function of the cyclone 208 according to the embodiment.

In the case of collecting viruses suspended in the air, such as an influenza virus, it is necessary to take in a large quantity of air and collect viruses in the taken air into a liquid because it is estimated that only a very small quantity of virus is suspended in the air. Here, the viruses are collected into the liquid to generally perform the above-mentioned binding between antibodies and virus components in the liquid. The liquid may be pure water free of impurities, or a solution prepared by dissolving in pure water a phosphate buffer typically used as a solvent of a biological material, so that the virus components are not degenerated. For example, phosphate buffered saline (PBS), Tris, and the like are available.

The cyclone 208 may be used to take in a large quantity of air. In the cyclone 208, as illustrated in FIG. 3(a), air is sucked through a suction port 281 connected to the suction device 202, and thereby the air is taken into the cyclone 208 through the air intake port 210. The taken air is rotated at a high speed in the cyclone 208. At this time, microparticles contained in the taken air and having a size larger than or equal to a certain size are unable to follow an air flow in the cyclone 208 and are centrifugally blown toward an inner wall surface of the cyclone 208, thereby being separated from the air. The microparticles separated from the air are collected to a lower portion of the cyclone 208.

In this way, the suction into the cyclone 208 causes an influenza virus suspended in the air to enter the cyclone 208 through the air intake port 210 and to be centrifugally blown toward the inner wall surface of the cyclone 208. In a case where the lower portion of the cyclone 208 is filled with a predetermined quantity of collection liquid 283 before starting the suction, an airflow in the cyclone 208 causes the collection liquid 283 to spirally rotate and to rise along the inner wall surface of the cyclone 208 as illustrated in FIG. 3(b), and an influenza virus blown toward the inner wall surface can be captured in the solution. The collection liquid 283 is supplied, for example, from a collection liquid intake port 282 of the cyclone 208 connected to the pump 206 into the cyclone 208.

The collection device 200 may collect a virus from a mucous membrane or mucus of a subject collected from the pharynx, nasal cavity, or the like of the subject by using a specimen collecting tool, such as a sterilized swab, or may collect a virus from mucus or the like collected by nasal aspiration, instead of collecting a virus from the air. Collecting of a virus by the collection device 200 is not limited to the above-described examples, and another method according to the related art may be used as long as a virus can be collected from a subject.

### Configuration of Detection Device

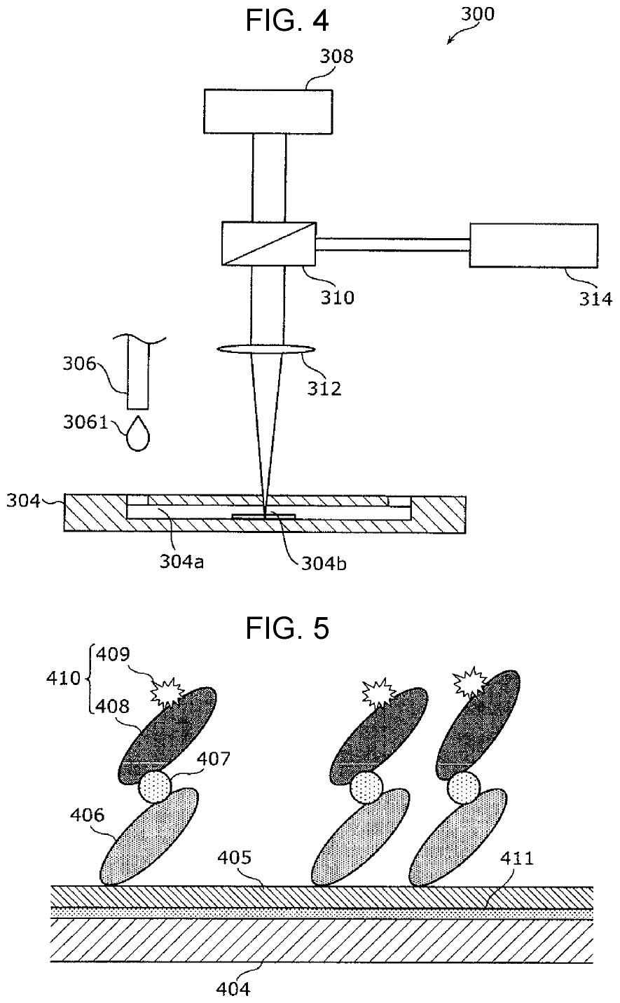

The detection device 300 will be described in detail with reference to FIG. 2 and FIG. 4. FIG. 4 is a configuration diagram of the detection device 300 according to the embodiment.

The detection device 300 detects the quantity of virus from a collection liquid mixed with microparticles by the collection device 200. As illustrated in FIG. 2 and FIG. 4, the detection device 300 includes a sensor device 302, a loading unit 306, a light source 308, a beam splitter 310, a lens 312, and a detecting unit 314. Hereinafter, the individual components of the detection device 300 will be described.

The sensor device 302 includes a sensor cell 304. In FIG. 2, the sensor device 302 includes the single sensor cell 304. Alternatively, the sensor device 302 may include sensor cells.

In the present embodiment, the sensor device 302 is capable of detecting a virus in a concentration range from 0.1 pM to 100 nM. In the present embodiment, a surface-enhanced fluorescence method is used to optically detect the quantity of virus. A virus concentration can be calculated by using a calibration curve created by measuring the fluorescence intensity of a sample whose virus concentration is known.

The sensor cell 304 generates surface plasmons when irradiated with excitation light, thereby enhancing fluorescence emitted by a fluorescent substance bound to a virus. As illustrated in FIG. 4, the sensor cell 304 includes a channel 304a and a detection region 304b.

The channel 304a is a path for leading a sample liquid 3061 dropped by the loading unit 306 to the detection region 304b.

The detection region 304b is a region for optically detecting a virus by using surface plasmons. A metal microstructure is disposed in the detection region 304b, where surface plasmons are generated when irradiated with excitation light emitted by the light source 308. In addition, first VHH antibodies are immobilized on the metal microstructure. The first VHH antibodies are immobilized antibodies that bind specifically to a virus. The details of the detection region 304b will be described below with reference to FIG. 4 and FIG. 5.

The loading unit 306 loads second VHH antibodies and a specimen to the sensor cell 304. Specifically, the loading unit 306 drops the sample liquid 3061 containing the second VHH antibodies and the specimen onto the sensor cell 304. The second VHH antibodies are labeled antibodies labeled with fluorescent substances. The specimen is a liquid that may contain a virus and is, in the present embodiment, a collection liquid discharged by the cyclone 208. The loading unit 306 may drop the specimen and then the second VHH antibody onto the sensor cell 304, instead of dropping the sample liquid 3061 containing the second VHH antibodies and the specimen onto the sensor cell 304.

If the specimen contains a virus, the virus binds to the metal microstructure via the first VHH antibodies. At this time, the virus also binds to the second VHH antibodies labeled with fluorescent substances. In other words, complexes each made up of a first VHH antibody, a virus, a second VHH antibody, and a fluorescent substance binds to the metal microstructure. When the metal microstructure is irradiated with light in this state, the fluorescent substances indirectly bound to the virus emit fluorescence, and the fluorescence is enhanced by surface plasmons. Hereinafter, the fluorescence enhanced by surface plasmons will be referred to as surface-enhanced fluorescence. When antibodies having a sufficiently high binding capacity with respect to a virus is used as the first VHH antibodies, a state where the labeled antibodies bound to the virus are bound to the first VHH antibodies can be made thermally more stable than a state where the labeled antibodies bound to the virus are not bound to the first VHH antibodies. Accordingly, more labeled antibodies bound to the virus can be bound to the first VHH antibodies and can be collected to the surface of the metal microstructure.

The light source 308 is an example of a light irradiator that irradiates the sensor cell 304 with excitation light. Any device according to the related art can be used as the light source 308 without particular limitation. For example, a laser, such as a semiconductor laser or a gas laser, can be used as the light source 308. The light source 308 may emit excitation light whose wavelength has a small interaction with a substance contained in a virus (for example, 400 nm to 2000 nm). Furthermore, the wavelength of the excitation light may be 600 nm to 850 nm that can be used by a semiconductor laser.

The beam splitter 310 separates the surface-enhanced fluorescence generated in the detection region 304b from the excitation light emitted by the light source 308. Specifically, the beam splitter 310 allows the excitation light from the light source 308 to pass therethrough, separates the surface-enhanced fluorescence generated in the sensor cell 304 from the excitation light, and leads the surface-enhanced fluorescence to the detecting unit 314.

The lens 312 condenses the excitation light emitted by the light source 308 and passed through the beam splitter 310 onto the detection region 304b.

The detecting unit 314 divides the surface-enhanced fluorescence led by the beam splitter 310 and detects light in a specific wavelength range, thereby outputting an electric signal corresponding to the quantity of virus in the specimen. Any device capable of detecting light in the specific wavelength range according to the related art can be used as the detecting unit 314 without particular limitation. For example, an interference filter that allows a specific wavelength range to pass therethrough to divide light, a Czerny spectrometer that divides light by using a diffraction grating, an Echelle spectrometer, or the like can be used as the detecting unit 314. Furthermore, the detecting unit 314 may include a notch filter for removing the excitation light from the light source 308, or a longpass filter that is capable of blocking the excitation light from the light source 308 and allowing the surface-enhanced fluorescence generated by the sensor cell 304 to pass therethrough.

In a case where a virus concentration is unknown, the time period taken for the detecting unit 314 to perform detection with highly accurate measurement tends to increase as the virus concentration decreases compared to a predetermined concentration. Thus, the detection device 300 may include a memory storing correlation data of detection values and virus concentrations, and the detecting unit 314 may output a virus concentration associated with a detection value in the correlation data stored in the memory. A detection value associated in the correlation data is a detection value when a predetermined time period elapses from the start of detection. Thus, the detecting unit 314 outputs a virus concentration associated in the correlation data with the detection value detected when the predetermined time period elapses from the start of detection. Accordingly, the detecting unit 314 is capable of outputting a virus concentration when the predetermined time period elapses that is shorter than a measurement time period in which the virus concentration can be accurately measured. Here, the detection device 300 calculates a virus concentration by using a detection value of the detecting unit 314 and the correlation data. Alternatively, the calculation of a virus concentration may be performed by another device, such as the controller 400. In this case, the other device includes a memory storing the correlation data.

The detection device 300 is not limited to the above-described example, and another method according to the related art may be used as long as a virus can be detected.

### Configuration of Controller

The controller 400 controls the operation of the entire pathogen detection apparatus 10. Specifically, the controller 400 controls the collection device 200, the detection device 300, and the display device 500. In addition, the controller 400 obtains a measurement result of a body temperature measured by the body temperature measurement device 100.

More specifically, the controller 400 controls the start of measurement, causes the suction device 202 to start sucking the surrounding air, and causes the pump 206 to supply a collection liquid from the collection liquid tank 204 to the cyclone 208. Accordingly, the collection liquid is mixed with microparticles in the cyclone 208, and a specimen is supplied from the cyclone 208 to the detection device 300. Furthermore, the controller 400 causes the light source 308 to emit light and causes the detecting unit 314 to detect surface-enhanced fluorescence.

For example, the controller 400 controls the collection device 200 and the detection device 300 in accordance with a body temperature measurement result output by the body temperature measurement device 100. In addition, the controller 400 causes the display device 500 to display a detection result obtained by the detection device 300. In addition, the controller 400 is capable of controlling each pump to supply a predetermined volume of sample liquid to the detection device 300 under a preset condition on the basis of an input parameter. Furthermore, the controller 400 may have a time measurement function, and may generate and store information on the time taken for each operation. In addition, the controller 400 may receive a measurement value from the detection device 300, and may calculate a chronological change in the concentration of a virus suspended in the air on the basis of the measurement value and time information.

The controller 400 is formed of, for example, one or more dedicated electronic circuits. The one or more dedicated electronic circuits may be integrated on one chip or may be individually formed on chips. Alternatively, the controller 400 may be formed of, instead of the one or more dedicated electronic circuits, a general-purpose processor (not illustrated) and a memory (not illustrated) storing a software program or instruction. In this case, the processor functions as the controller 400 when the software program or instruction is executed.

Configuration of Display Device

The display device 500 displays information, such as a body temperature measurement result or a virus detection result. The display device 500 is, for example, a liquid crystal display, an organic electroluminescence (EL) display, electronic paper, or the like. The display device 500 is not limited to the above examples, and another display device according to the related art may be used as long as information can be displayed.

Next, a detection method for the detection device 300 will be described in detail.

One influenza virus contains virus components including about 1000 nucleoprotein (NP) molecules. Thus, to detect a larger number of NP molecules to facilitate a detection, a pretreatment of crushing an influenza virus and extracting the NP molecules contained in the influenza virus may be performed in advance, for example, before causing a pathogen to react with an antibody. To crush the influenza virus, a surface-active agent is injected to break a membrane substance that covers the surface of the influenza virus, and the NP molecules therein are extracted. As the surface-active agent used for crush, Tween 20, Triton X, Sarkosyl, and the like are available. Alternatively, a captured virus may be caused to react with an antibody for detection without crushing the virus.

The pretreatment may include, in addition to the above-described crush, any of a process of removing foreign substances, a process of concentrating a virus or virus components, and a process of labeling a virus or virus components with a labeled substance used for detection, such as a fluorescent substance or a magnetic substance. The pretreatment is not limited to the above examples as long as detection of a pathogen is promoted. The process for promoting detection of a pathogen may be a process for efficiently detecting the quantity of pathogen or a process for accurately detecting the quantity of pathogen.

In general, detection of a biological material is performed by using an antigen-antibody reaction in which an antigen is caused to react with an antibody. Here, the antigen is an influenza virus or NP, which is a component contained in the influenza virus. The antibody reacts specifically with the antigen and binds to the antigen. Hereinafter, a detection method using an antigen-antibody reaction will be described in detail.

A description will be given with reference to FIG. 5. FIG. 5 is a diagram for describing the details of an antigen-antibody reaction.

First, on a surface of a substrate 404 disposed in the above-described sensor cell 304, first antibodies 406 are formed which bind to a virus or NP as a virus component serving as an antigen. The first antibodies 406 play a role in capturing NP molecules 407 or the like to the surface of the substrate 404. The first antibodies 406 are, for example, IgG antibodies. Among IgG antibodies, those having an ability to bind specifically to an influenza virus or NP as an influenza virus component may be used. The first antibodies 406 are also referred to as capture antibodies. The surface of the substrate 404 is modified with a self-assembled monolayer (SAM) 405 to cause the inorganic substrate and the organic antibodies to bind to each other. The first antibodies 406 are immobilized on the surface of the substrate 404 via the SAM 405.

The SAM 405 is formed on a surface of a gold single-crystal thin layer 411 formed on the surface of the substrate 404. Accordingly, the SAM 405 is a closely-packed and regularly-oriented monolayer formed by the Au—S—R bond resulting from alkanethiol (R—SH) bound to the single-crystal thin layer 411. In this way, in the antigen-antibody reaction, the first antibodies 406 are caused to bind to the SAM 405 formed on the surface of the substrate 404.

Subsequently, a solution containing the NP molecules 407, which are antigens, is injected to the first antibodies 406 immobilized on the surface of the substrate 404. In other words, a solution containing the NP molecules 407 is injected to the detection region 304b of the sensor cell 304. At this time, the first antibodies 406 start binding to the NP molecules 407 as antigens, and then the number of bonds increases as time elapses. While the number of bonds increases, dissociation occurs. Accordingly, the first antibodies 406 and the NP molecules 407 repeat binding and dissociation to reach an equilibrium state.

Subsequently, a solution containing second antibodies 408 is injected to the detection region 304b of the sensor cell 304. Like the first antibodies 406, the second antibodies 408 are, for example, IgG antibodies capable of binding to an influenza virus or the NP molecules 407, which are influenza virus components. A labeled substance 409 that emits a signal for performing detection is bound to each second antibody 408 in advance. The labeled substance 409 may be, for example, a substance that emits fluorescence when being irradiated with laser light having a predetermined wavelength. The labeled substance 409 is, for example, DyLight 800 that emits fluorescence having a wavelength of 800 nm when being irradiated with laser light having a wavelength of 785 nm. The second antibody 408 to which the labeled substance 409 is bound is also referred to as a labeled antibody 410.

In a case where a virus is present in the air, the virus is captured into the collection liquid 283 in the cyclone 208 when the cyclone 208 is operated. The captured virus is crushed, and thereby the NP molecules 407 in the virus are extracted. When a solution containing the NP molecules 407 obtained accordingly is injected to the surface of the substrate 404 by being injected to the detection region 304b of the sensor cell 304, the NP molecules 407 bind to the first antibodies 406 serving as capture antibodies formed on the surface of the substrate 404. Furthermore, when a solution containing the second antibodies 408 serving as labeled antibodies each bound to the labeled substance 409 that emits fluorescence is injected, the second antibodies 408 bind to the NP molecules 407, which are antigens bound to the first antibodies 406. The binding of the first antibodies 406, the NP molecules 407 as antigens, and the second antibodies 408 is referred to as sandwich assay. The solution in the detection region 304b that has undergone sandwich assay is irradiated with laser light, which is excitation light for exciting fluorescence in the labeled substances 409 bound to the second antibodies 408, and the excited fluorescence is measured to obtain a signal to be detected.

In the detection device 300, the light source 308 repeatedly emits laser light at predetermined intervals, and the detecting unit 314 repeatedly detects, at predetermined intervals, fluorescence excited from the labeled substances 409 in response to irradiation with the laser light. The repeated irradiation with laser light increases the intensity of emitted fluorescence as the binding of the first antibodies 406, the NP molecules 407, and the labeled antibodies 410 progresses. When a solution containing the labeled antibodies 410 is injected, a labeled antibody 410 that does not bind to any NP molecule 407 is suspended in a liquid layer. The number of bonds between the NP molecules 407 and the labeled antibodies 410 changes in accordance with the amount of solution injected and/or the thickness of the liquid layer held in the sensor cell 304.

In an early stage after the solution of the labeled antibodies 410 is injected, the number of bonds between the NP molecules 407 and the labeled antibodies 410 gradually increases. When the intensity of laser light that excites fluorescence in the labeled substances 409 of the labeled antibodies 410 is increased, the labeled substance 409 of a suspended labeled antibody 410 that is not bound to any NP molecule 407 emits light. If the fluorescence emitted at this time is detected, it is not possible to accurately detect the NP molecules 407, and thus it is not possible to indiscriminately increase the intensity of laser light. On the other hand, when the quantity of virus in the air is very small, a small quantity of NP molecule 407 is obtained, and thus the intensity of excitation light may be increased.

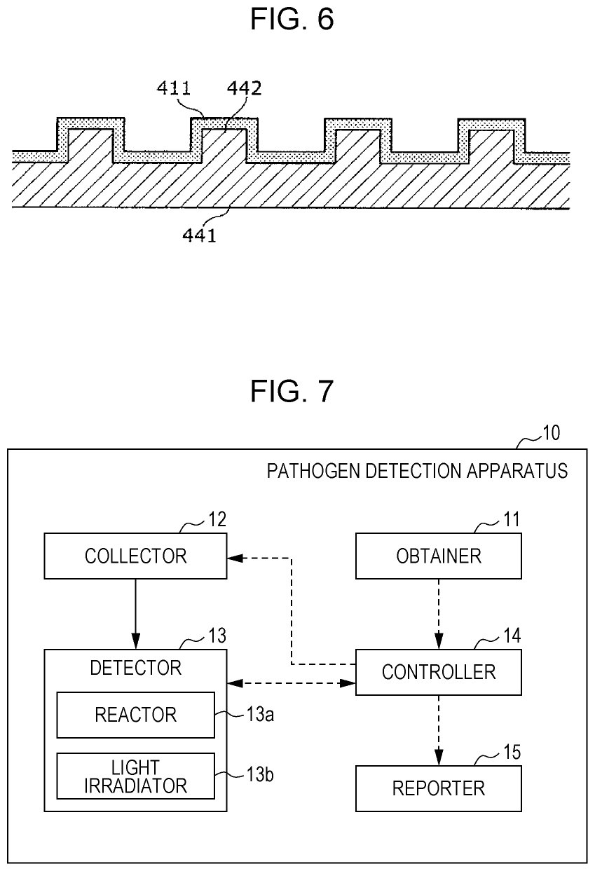

To increase the strengths of signals from the labeled antibodies 410 bound to the NP molecules 407 near the surface of the substrate 404, surface plasmon resonance is used. FIG. 6 is a diagram illustrating an example of a substrate structure in the case of using surface plasmon resonance.

Surface plasmon resonance has traditionally been known. For example, as illustrated in FIG. 6, nano-size protrusions 442 are formed on a surface of a substrate 441, and a single-crystal thin layer 411 made of Au or the like is formed on the surfaces of the protrusions 442, and thereby a strong-electromagnetic-field region is formed near the surface of the substrate 441. The strong-electromagnetic-field region is formed very close to the surface of the substrate 441, which enables the labeled substance 409 emitting a signal of the second antibody 408 bound to the NP molecule 407 to emit light whose intensity is higher than the light emitted by the labeled substance 409 of the labeled antibody 410 that is suspended away from the surface of the substrate 441 and is not bound to the NP molecule 407. The combination of surface plasmon resonance and sandwich assay makes it possible to effectively detect a very small quantity of virus in the air and to effectively detect a transient state where the signal strength gradually increases in an early stage after the second antibodies 408 and the NP molecules 407 start binding to each other.

Next, the functional configuration of the pathogen detection apparatus 10 will be described.

FIG. 7 is a block diagram illustrating an example of the functional configuration of the pathogen detection apparatus 10 according to the embodiment.

As illustrated in FIG. 7, the pathogen detection apparatus 10 includes an obtainer 11, a collector 12, a detector 13, a controller 14, and a reporter 15.

The obtainer 11 obtains a body temperature of a subject. The obtainer 11 obtains a body temperature of a subject by measuring the body temperature of the subject. The obtainer 11 is implemented by, for example, the body temperature measurement device 100.

The collector 12 collects a pathogen carried by the subject or a pathogen in air around the subject. The pathogen is a virus, for example, an influenza virus. The collector 12 discharges a specimen, which is obtained by mixing the pathogen into a collection liquid, to the detector 13. The collector 12 is implemented by, for example, the collection device 200. To collect a pathogen carried by the subject, for example, a sampling tube to be inserted into the mouth of the subject to collect exhaled breath is used. To collect a pathogen in the air around the subject, for example, a cyclone is used.

The detector 13 detects the pathogen collected by the collector 12. Specifically, the detector 13 includes a reactor 13a and a light irradiator 13b.

The reactor 13a causes a reaction to occur between the pathogen collected by the collector 12 and the labeled substances 409. For example, the reactor 13a causes the first antibodies 406, the NP molecules 407, and the second antibodies 408 to which the labeled substances 409 bound to react with each other, thereby causing them to bind to each other. In this way, the “reaction between a pathogen and a labeled substance” includes an indirect reaction between a pathogen and a labeled substance, that is, a reaction between a pathogen and a substance (antibody) bound to a labeled substance. The reaction in the reactor 13a is not limited to a reaction using surface plasmon resonance, and any reaction involving binding between a pathogen and the labeled substances 409 may be performed. The reactor 13a is implemented by, for example, the sensor cell 304 of the detection device 300.

The light irradiator 13b irradiates, with excitation light, a reacted substance (i.e., a specimen) obtained through the reaction in the reactor 13a. The light irradiator 13b is implemented by, for example, the light source 308.

Accordingly, the detector 13 detects the pathogen on the basis of fluorescence generated by the labeled substances 409 as a result of irradiation with the excitation light. The detector 13 may detect the intensity of fluorescence generated by the labeled substances 409 as a result of irradiation with the excitation light, and may detect whether or not the subject is infected with the pathogen in accordance with whether or not the detected intensity of fluorescence is higher than a predetermined threshold value. Specifically, in a case where the detected intensity of fluorescence is higher than the predetermined threshold value, the detector 13 may detect that the subject is infected with the pathogen, and, in a case where the detected intensity of fluorescence is lower than or equal to the predetermined threshold value, the detector 13 may detect that the subject is not infected with the pathogen.

The detector 13 may detect the quantity of labeled substance on the basis of the detected intensity of fluorescence, and correlation data between the intensity of fluorescence and the quantity of labeled substance stored in advance. On the basis of the intensity of fluorescence detected when a predetermined time period elapses from the start of reaction in the reactor 13a, that is, from the start of detection, and the correlation data, the detector 13 may specify the quantity of labeled substance associated with the detected intensity of fluorescence in the correlation data, and may output the specified quantity of labeled substance. The quantity of labeled substance corresponds to the number of pathogens or a pathogen concentration. The correlation data may include the correlations between the intensity of fluorescence and the quantity of labeled substance at the elapse of two or more different time periods.

The detector 13 may perform a pretreatment for promoting detection on the pathogen collected by the collector 12.

The detector 13 is implemented by, for example, the detection device 300, the controller 400, and the like.

The controller 14 controls at least one of the collector 12 or the detector 13 to shorten the time period from the start of collection by the collector 12 to the report of a detection result by the reporter 15 in a case where the body temperature of the subject obtained by the obtainer 11 is higher than a predetermined threshold value. Specifically, in a case where the body temperature of the subject obtained by the obtainer 11 is higher than the predetermined threshold value, the controller 14 controls at least one of the collector 12 or the detector 13 in a first control mode. In a case where the body temperature is lower than or equal to the predetermined threshold value, the controller 14 controls at least one of the collector 12 or the detector 13 in a second control mode. In the second control mode, the time period from the start of pathogen collection to the report of a detection result is shorter than in the first control mode.

For example, the controller 14 may control the detector 13 such that the time period from the start of pathogen collection to the report of a detection result in the second control mode is shorter than in the first control mode. Specifically, in a case where the body temperature of the subject obtained by the obtainer 11 is lower than or equal to the predetermined threshold value, the controller 14 controls the detector 13 in a first detection mode in which the detector 13 detects the pathogen for a first time period. In a case where the body temperature of the subject is higher than the predetermined threshold value, the controller 14 controls the detector 13 in a second detection mode in which the detector 13 detects the pathogen for a second time period shorter than the first time period.

In the first detection mode, the detector 13 detects the pathogen, for example, a virus, on the basis of fluorescence generated by a labeled substance as a result of irradiating, with excitation light, a reacted substance obtained through the reaction for the first time period. In the second detection mode, the detector 13 detects the pathogen, for example, a virus, on the basis of fluorescence generated by a labeled substance as a result of irradiating, with excitation light, a reacted substance obtained through the reaction for the second time period shorter than the first time period.

In the first detection mode, the detector 13 may detect a pathogen concentration, for example, a virus concentration, on the basis of the intensity of fluorescence generated by a labeled substance as a result of irradiating, with excitation light, the detection region 304b for the first time period.

In the second detection mode, the detector 13 may detect a pathogen concentration, for example, a virus concentration, on the basis of the intensity of fluorescence generated by a labeled substance as a result of irradiating, with excitation light, the detection region 304b for the second time period shorter than the first time period.

The controller 14 is implemented by, for example, the controller 400.

The reporter 15 reports a detection result obtained by the detector 13. For example, the reporter 15 may display a detection result indicating whether or not the subject is infected with the pathogen or may display a detection result indicating the detected quantity of labeled substance of the pathogen. The reporter 15 is implemented by, for example, the display device 500.

The reporter 15 may report a detection result by using a sound, by printing a printed matter, or by turning on a light source, such as a light emitting diode (LED), instead of displaying the detection result.

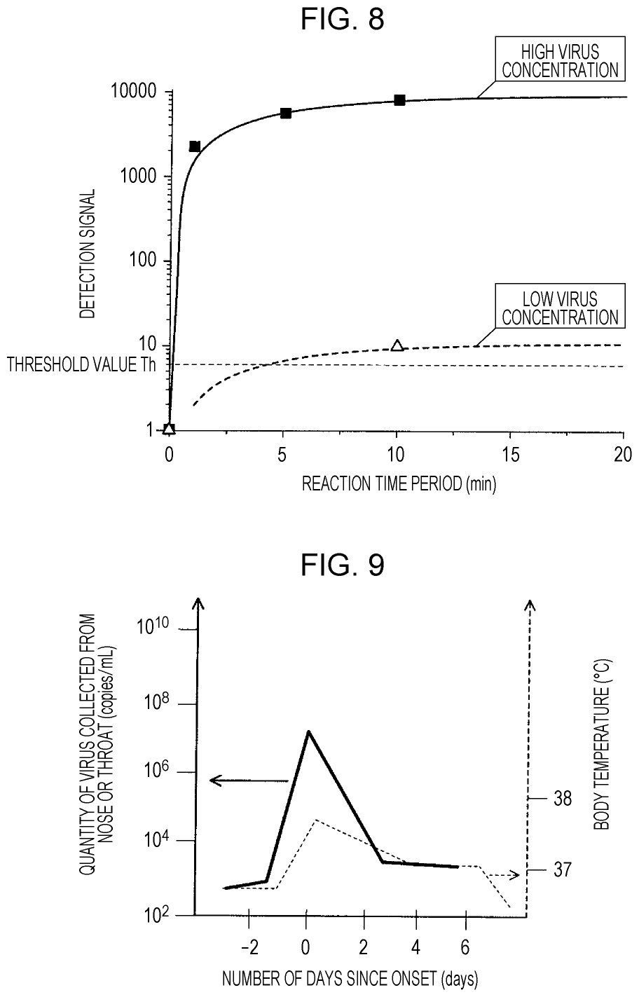

Now, the relationship between a reaction time period and a detection signal in different virus concentrations will be described with reference to FIG. 8. In FIG. 8, the horizontal axis represents the reaction time period, which is a time period from the start of the reaction in the reactor 13a, and the vertical axis represents the detection signal, which indicates the intensity of fluorescence detected by the detector 13. A reaction start time may be, for example, a time at which a pathogen and the labeled substances 409 are loaded to the sensor cell 304.

As illustrated in FIG. 8, in a case where the virus concentration is high, a large value of the detection signal is detected even if the reaction time period is short, for example, about 1 minute. It is understood that, in a case where the virus concentration is high, for example, the value of the detection signal is larger than a threshold value Th of the detection signal before 1 minute elapses from the start of reaction. The threshold value Th is used as a reference for determining whether the subject is infected with the virus. The threshold value Th of the detection signal is set to, for example, a value larger than a noise level detected by the detector 13 in full darkness, for example.

On the other hand, in a case where the virus concentration is low, the value of the detection signal exceeds the threshold value Th after a long reaction time period elapses, for example, more than 5 minutes. Thus, it is necessary to perform detection after waiting for a long reaction time period, for example, 10 minutes.

Therefore, in a case where a high virus concentration is expected, whether the subject is infected with the virus can be determined even if the reaction time period is short. In other words, in a case where there is a possibility that the virus concentration is high, whether the subject is infected with the virus can be detected even if the time period for detection is shorter than in a case where there is a possibility that the virus concentration is low.

Next, the relationship between the quantity of virus carried by a subject and a body temperature of the subject will be described with reference to FIG. 9. In FIG. 9, the vertical axis on the left represents the quantity of virus collected from the nose or throat of the subject, the vertical axis on the right represents the body temperature of the subject, and the horizontal axis represents the number of days since onset, that is, since when a symptom such as cough or running nose caused by the virus appears. The day of onset is represented by 0.

It is understood from FIG. 9 that there is a positive correlation between the quantity of virus and the body temperature. Accordingly, it can be estimated that, in a case where the body temperature of the subject is higher than a predetermined threshold value, there is a high possibility that the subject carries the virus in high concentration. The predetermined threshold value is a body temperature higher than a normal body temperature of the subject and may be, for example, 37° C. The predetermined threshold value may be set to a different value according to a subject and may be, for example, a value obtained by adding 1° C. to a normal body temperature. For example, when the normal body temperature of a subject is 36° C., 37° C. may be set as the predetermined threshold value, and when the normal body temperature of a subject is 36.5° C., 37.5° C. may be set as the predetermined threshold value.

Referring back to FIG. 8, the reaction in the reactor 13a reaches equilibrium as the reaction time period elapses regardless of the virus concentration. Thus, the detection signal obtained by irradiating a reacted substance with excitation light tends to be constant. The virus concentration may be calculated by using the detection signal that has become constant, but time is taken until the detection signal becomes constant. Thus, in a case where there is a possibility that the virus concentration is high, the detector 13 calculates the virus concentration by using the detection signal, that is, the intensity of fluorescence, and correlation data, when the second time period elapses. The correlation data has data indicating the correspondence between the intensity of fluorescence and the quantity of labeled substance when the second time period elapses in a case where the virus concentration is high. Also, in a case where there is a possibility that the virus concentration is low, the detector 13 may calculate the virus concentration by using the detection signal, that is, the intensity of fluorescence and correlation data, when the first time period elapses. The correlation data may have data indicating the correspondence between the intensity of fluorescence and the quantity of labeled substance when the first time period elapses in a case where the virus concentration is low.

### Operation of Pathogen Detection Apparatus

Next, a pathogen detection method for the pathogen detection apparatus 10 will be described.

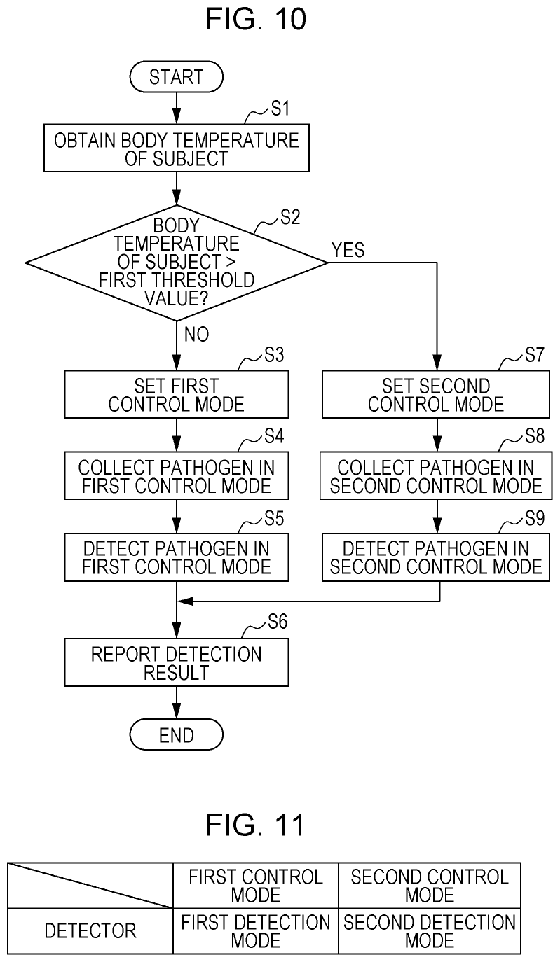

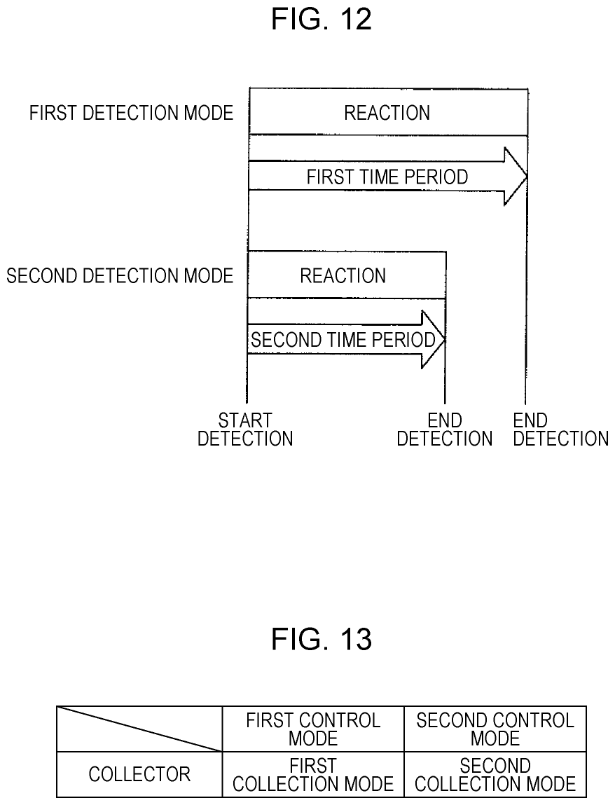

FIG. 10 is a flowchart illustrating an example of a pathogen detection method for the pathogen detection apparatus 10 according to the present embodiment. FIG. 11 is a diagram for describing the first control mode and the second control mode in the pathogen detection apparatus 10 according to the present embodiment. FIG. 12 is a diagram for describing the first detection mode and the second detection mode in the pathogen detection apparatus 10 according to the present embodiment.

As illustrated in FIG. 10, in the pathogen detection apparatus 10, the obtainer 11 obtains a body temperature of a subject (S1).

The controller 14 determines whether or not the body temperature of the subject obtained by the obtainer 11 is higher than a first threshold value (S2). The first threshold value is the predetermined threshold value described above.

In a case where the body temperature of the subject obtained by the obtainer 11 is lower than or equal to the first threshold value (NO in S2), the controller 14 sets the first control mode (S3). Accordingly, the controller 14 controls the detector 13 in the first detection mode, as illustrated in FIG. 11.

Subsequently, in the first control mode, the collector 12 collects a pathogen carried by the subject or a pathogen in the air around the subject (S4).

Subsequently, in the first control mode, the detector 13 detects the pathogen collected in the collection (S5). Specifically, as illustrated in FIG. 12, in the first detection mode, the light irradiator 13b irradiates the reactor 13a with excitation light for the first time period, and the detector 13 detects a pathogen concentration on the basis of the intensity of fluorescence when the first time period elapses from the start of detection (i.e., the start of irradiation with excitation light). At this time, the detector 13 may detect the quantity of labeled substance reacted with the pathogen.

In the first detection mode, the light irradiator 13b may irradiate the reactor 13a with excitation light for the first time period, and the detector 13 may detect a pathogen concentration on the basis of the intensity of fluorescence when the first time period elapses from the start of detection (i.e., the start of irradiation with excitation light).

On the other hand, in a case where the body temperature of the subject obtained by the obtainer 11 is higher than the first threshold value (YES in S2), the controller 14 sets the second control mode (S7). Accordingly, the controller 14 controls the detector 13 in the second detection mode, as illustrated in FIG. 11.

Subsequently, in the second control mode, the collector 12 collects a pathogen carried by the subject or a pathogen in the air around the subject (S8). Note that the operation of the collector 12 is the same both in the first control mode and the second control mode.

Subsequently, in the second control mode, the detector 13 detects the pathogen collected in the collection (S9). Specifically, as illustrated in FIG. 12, in the second detection mode, the detector 13 causes the reaction in the reactor 13a to be performed for the second time period shorter than the first time period, and detects the pathogen on the basis of the intensity of fluorescence when the second time period elapses from the start of detection (i.e., the start of reaction). At this time, the detector 13 may detect the quantity of labeled substance reacted with the pathogen.

The reporter 15 reports a detection result obtained by the detector 13 in step S5 or step S9 (S6).

### Advantages and the Like

In the pathogen detection apparatus 10 according to the above-described embodiment, in a case where the body temperature of the subject is higher than the predetermined threshold value, the controller 14 determines that there is a high possibility that the subject is infected with a pathogen exceeding a predetermined concentration, and determines that the pathogen is likely to be detected from the subject or the space around the subject. Thus, in a case where there is a high possibility that the subject is infected with a pathogen, the detector 13 may detect the pathogen even if the time period until a detection result is obtained is shorter than in a case where there is a low possibility that the subject is infected with the pathogen. Thus, the pathogen detection apparatus 10 according to the present embodiment is capable of efficiently detecting a pathogen from a subject or a space around the subject.

In the pathogen detection apparatus 10, in a case where the body temperature of the subject obtained by the obtainer 11 is lower than or equal to the predetermined threshold value, the controller 14 controls the detector 13 in the first detection mode in which the detector 13 detects a pathogen for the first time period. In a case where the body temperature of the subject is higher than the predetermined threshold value, the controller 14 controls the detector 13 in the second detection mode in which the detector 13 detects a pathogen for the second time period shorter than the first time period.

Accordingly, in a case where the body temperature of the subject is higher than the predetermined threshold value, the controller 14 determines that there is a high possibility that the subject is infected with a pathogen exceeding a predetermined concentration, and takes a shorter time period for detection than in a case where the body temperature of the subject is lower than or equal to the predetermined threshold value. In other words, in this case, the detector 13 is more likely to detect a pathogen even if the time period for detection is shortened than in a case where the body temperature of the subject is lower than or equal to the predetermined threshold value. Thus, the pathogen detection apparatus 10 according to the present embodiment is capable of efficiently detecting a pathogen from a subject or a space around the subject.

In the pathogen detection apparatus 10, in the first detection mode, the detector 13 detects a pathogen on the basis of fluorescence generated by a labeled substance as a result of irradiating, with excitation light, a reacted substance obtained through reaction for the first time period. In the second detection mode, the detector 13 detects a pathogen on the basis of fluorescence generated by a labeled substance as a result of irradiating, with excitation light, a reacted substance obtained through reaction for the second time period shorter than the first time period.

Accordingly, in a case where the body temperature of the subject is higher than the predetermined threshold value, the controller 14 determines that there is a high possibility that the subject is infected with a pathogen exceeding a predetermined concentration, and takes a shorter time period for reaction in detection than in a case where the body temperature of the subject is lower than or equal to the predetermined threshold value. In other words, in this case, the detector 13 is more likely to detect a pathogen even if the time period for reaction in detection is shortened than in a case where the body temperature of the subject is lower than or equal to the predetermined threshold value. Thus, the pathogen detection apparatus 10 according to the present embodiment is capable of efficiently detecting a pathogen from a subject or a space around the subject.

### First Modification Example

In the above-described embodiment, the controller 14 controls the detector 13 such that the time period from the start of pathogen collection to the report of a detection result is shorter in the second control mode than in the first control mode. However, the embodiment is not limited thereto. For example, the controller 14 may control the collector 12 such that the time period from the start of pathogen collection to the report of a detection result is shorter in the second control mode than in the first control mode. This is because, in a case where the body temperature of the subject is high and there is a high possibility that the subject is infected with a pathogen, it is estimated that a pathogen concentration is high in the breath exhaled by the subject or the air around the subject. In other words, in this case, it is considered that sufficient air for detecting a pathogen can be collected even if a short time period is taken for the collection.

In this case, unlike in the above-described embodiment, the controller 14 controls the collector 12 in a first collection mode in the first control mode, and controls the collector 12 in a second collection mode in the second control mode, as illustrated in FIG. 13. Note that the operation of the detector 13 is the same both in the first control mode and the second control mode.

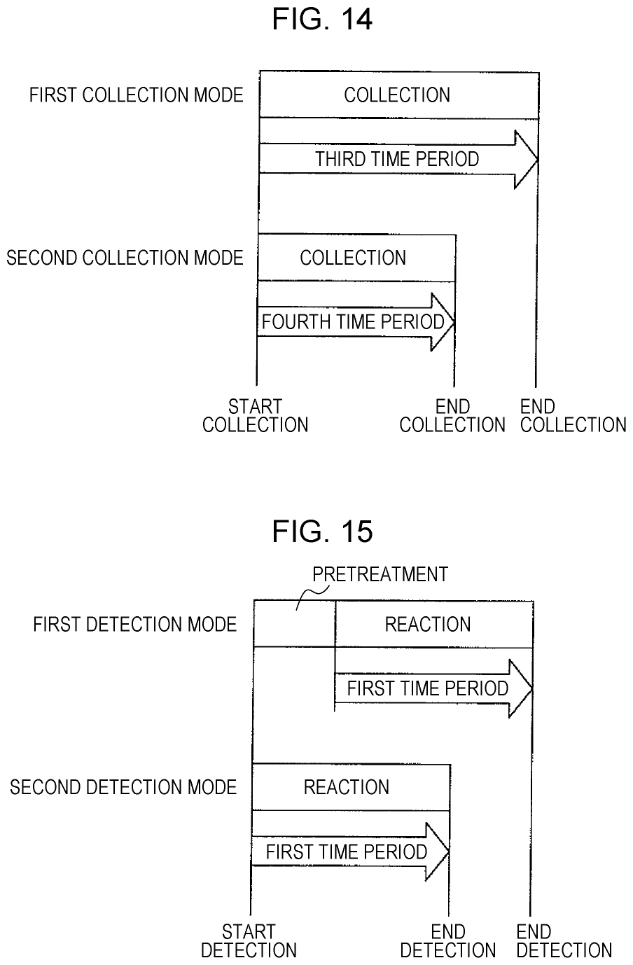

In the first modification example, as illustrated in FIG. 14, in a case where the body temperature of the subject obtained by the obtainer 11 is lower than or equal to the predetermined threshold value, the controller 14 controls the collector 12 in the first collection mode in which the collector 12 collects a pathogen for a third time period. In a case where the body temperature of the subject is higher than the predetermined threshold value, the controller 14 controls the collector 12 in the second collection mode in which the collector 12 collects a pathogen for a fourth time period shorter than the third time period.

FIG. 13 is a diagram for describing the first control mode and the second control mode in the pathogen detection apparatus 10 according to the first modification example. FIG. 14 is a diagram for describing the first collection mode and the second collection mode in the pathogen detection apparatus 10 according to the first modification example.

According to these figures, in a case where the body temperature of the subject is higher than the predetermined threshold value, the controller 14 determines that there is a high possibility that the subject is infected with a pathogen exceeding a predetermined concentration, and takes a shorter time period for collection than in a case where the body temperature of the subject is lower than or equal to the predetermined threshold value. In other words, in this case, the detector 13 is more likely to detect a pathogen even if the time period for collection by the collector 12 is shortened than in a case where the body temperature of the subject is lower than or equal to the predetermined threshold value. Thus, the pathogen detection apparatus 10 according to the first modification example is capable of efficiently detecting a pathogen from a subject or a space around the subject.

The first modification example may be combined with the embodiment. In other words, in the first control mode, the controller 14 may cause the collector 12 to operate in the first collection mode and may cause the detector 13 to operate in the first detection mode. In the second control mode, the controller 14 may cause the collector 12 to operate in the second collection mode and may cause the detector 13 to operate in the second detection mode.

### Second Modification Example

In the above-described embodiment, the controller 14 adjusts the time period from the start of reaction to detection in the detector 13 such that the time period from the start of pathogen collection to the report of a detection result is shorter in the second control mode than in the first control mode. However, the embodiment is not limited thereto. For example, the controller 14 may cause a pretreatment to be performed before reaction in the first control mode and may cause a pretreatment to be omitted in the second control mode, such that the time period from the start of pathogen collection to the report of a detection result is shorter in the second control mode than in the first control mode. This is because it is considered that, in a case where the body temperature of the subject is high and there is a high possibility that the subject is infected with a pathogen, a pathogen in high concentration can be obtained and thus the pathogen can be detected even if a pretreatment is not performed. As described above, the pretreatment is a process for promoting detection of a pathogen. Specific examples of the pretreatment have been described above.

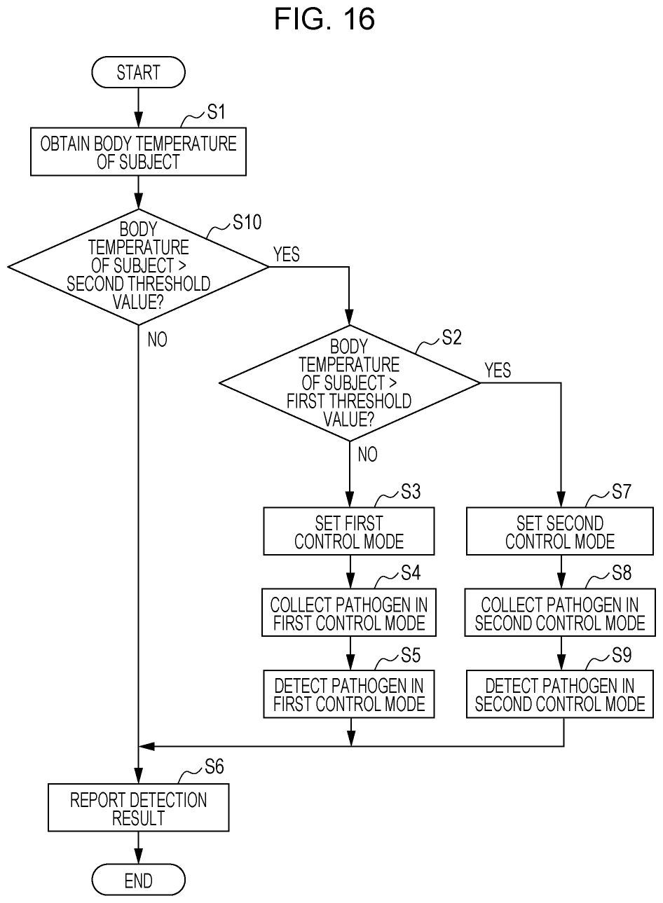

In this case, as illustrated in FIG. 15, the detector 13 performs a pretreatment before the reaction in the reactor 13a in the first detection mode in the first control mode, and omits a pretreatment before the reaction in the reactor 13a in the second detection mode in the second control mode. In the second modification example, the detector 13 detects a pathogen on the basis of the intensity of fluorescence when the first time period elapses from the start of reaction in both the first detection mode and the second detection mode.

In other words, in the second modification example, in a case where the body temperature of the subject obtained by the obtainer 11 is lower than or equal to the first threshold value, the controller 14 causes the detector 13 to perform a pretreatment in detection. In a case where the body temperature of the subject is higher than the first threshold value, the controller 14 causes the detector 13 to omit a pretreatment in detection.

FIG. 15 is a diagram for describing the first detection mode and the second detection mode in the pathogen detection apparatus 10 according to the second modification example.

According to the figure, in a case where the body temperature of the subject is higher than the predetermined threshold value, the controller 14 determines that there is a high possibility that the subject is infected with a pathogen exceeding a predetermined concentration, and shortens the time period for detection by omitting a pretreatment in detection. In other words, in this case, the detector 13 is more likely to detect a pathogen even if a pretreatment is omitted than in a case where the body temperature of the subject is lower than or equal to the predetermined threshold value. Thus, the pathogen detection apparatus 10 according to the second modification example is capable of efficiently detecting a pathogen from a subject or a space around the subject.

The second modification example may be combined with the embodiment. In other words, in the first control mode, the controller 14 may cause the detector 13 to operate in the first detection mode to perform a pretreatment and then detect a pathogen on the basis of the intensity of fluorescence when the first time period elapses. In the second control mode, the controller 14 may cause the detector 13 to operate in the second detection mode to omit a pretreatment and detect a pathogen on the basis of the intensity of fluorescence when the second time period elapses.

### Third Modification Example

In the above-described embodiment, the controller 14 changes the control mode by comparing the body temperature of the subject with one threshold value. The embodiment is not limited thereto, and the controller 14 may change the control mode by comparing the body temperature of the subject with two threshold values.

FIG. 16 is a flowchart illustrating an example of a pathogen detection method for the pathogen detection apparatus 10 according to a third modification example.

The pathogen detection method according to the third modification example is different from the pathogen detection method according to the embodiment in that step S10 is further performed in which the controller 14 determines whether or not the body temperature of the subject is higher than a second threshold value. The second threshold value is smaller than the first threshold value and is, for example, a normal body temperature of the subject.

In a case where the body temperature of the subject is lower than or equal to the second threshold value (NO in S10), the controller 14 proceeds to step S6. The reporter 15 reports a detection result indicating that no pathogen has been detected (S6).

On the other hand, in a case where the body temperature of the subject is higher than the second threshold value (YES in S10), the controller 14 performs step S2 and the subsequent steps in the pathogen detection method according to the embodiment. The detailed description thereof is omitted here.

### Fourth Modification Example

In the third modification example, in a case where the body temperature of the subject is lower than or equal to the second threshold value, the reporter 15 reports a detection result indicating that no pathogen has been detected. The embodiment is not limited thereto. In a case where the body temperature of the subject is lower than or equal to the second threshold value, the controller 14 may control the collector 12 and the detector 13 in a third control mode.

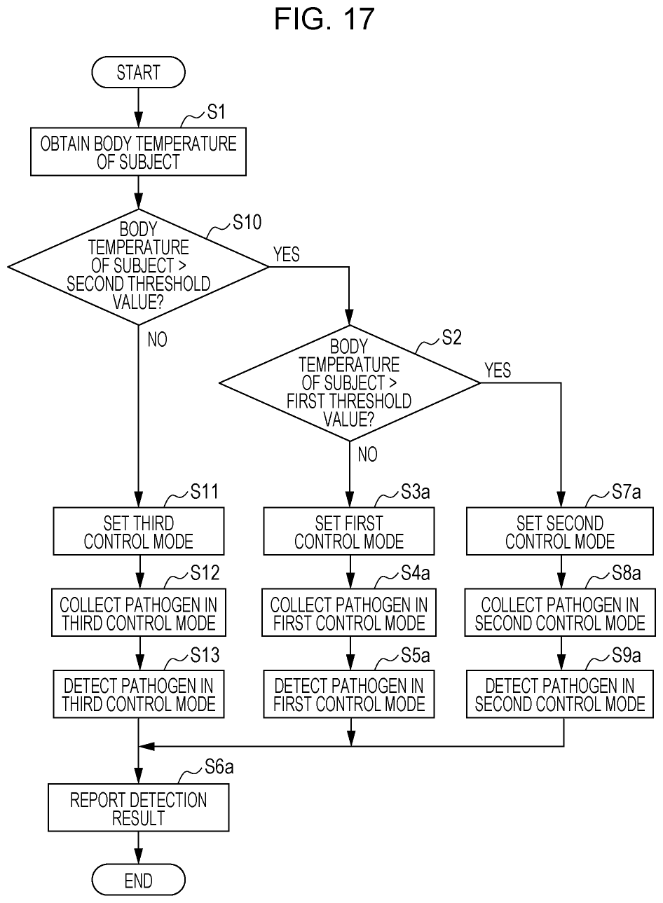

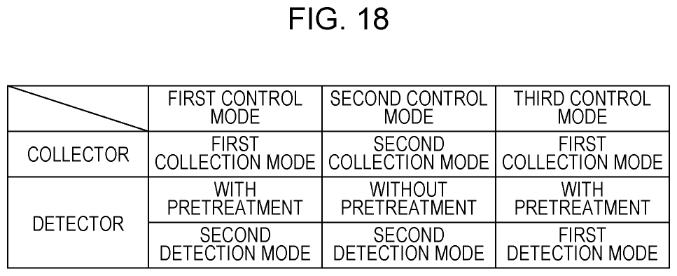

FIG. 17 is a flowchart illustrating an example of a pathogen detection method for the pathogen detection apparatus 10 according to a fourth modification example. FIG. 18 is a diagram for describing the first to third control modes in the pathogen detection apparatus 10 according to the fourth modification example.

As illustrated in FIG. 17, in the pathogen detection apparatus 10, the obtainer 11 obtains a body temperature of a subject (S1).

The controller 14 determines whether or not the body temperature of the subject obtained by the obtainer 11 is higher than a second threshold value (S10). Here, the second threshold value is the same as the second threshold value in the third modification example.

In a case where the body temperature of the subject obtained by the obtainer 11 is lower than or equal to the second threshold value (NO in S10), the controller 14 sets the third control mode (S11). Accordingly, the controller 14 controls the collector 12 in the first collection mode and controls the detector 13 in the first detection mode with a pretreatment, as illustrated in FIG. 18.

Subsequently, in the third control mode, the collector 12 collects a pathogen carried by the subject or a pathogen in the air around the subject (S12). Specifically, the collector 12 collects a pathogen for the third time period in the first collection mode, as described above with reference to FIG. 14.

Subsequently, in the third control mode, the detector 13 detects the pathogen collected in the collection (S13). Specifically, the detector 13 performs a pretreatment, causes the reaction in the reactor 13a to be performed for the first time period in the first detection mode as illustrated in FIG. 12, and detects the pathogen on the basis of the intensity of fluorescence when the first time period elapses from the start of detection (i.e., the start of reaction). At this time, the detector 13 may detect the quantity of labeled substance reacted with the pathogen.

Going back to step S10, in a case where the body temperature of the subject obtained by the obtainer 11 is higher than the second threshold value (YES in S10), the controller 14 determines whether or not the body temperature of the subject is higher than a first threshold value (S2). Here, the first threshold value is the same as the first threshold value according to the third modification example.

In a case where the body temperature of the subject obtained by the obtainer 11 is lower than or equal to the first threshold value (NO in S2), the controller 14 sets the first control mode (S3a). Accordingly, the controller 14 controls the collector 12 in the first collection mode and controls the detector 13 in the second detection mode with a pretreatment, as illustrated in FIG. 18.

Subsequently, in the first control mode, the collector 12 collects a pathogen carried by the subject or a pathogen in the air around the subject (S4a). Specifically, the collector 12 collects a pathogen for the third time period in the first collection mode, as in step S12.

Subsequently, in the first control mode, the detector 13 detects the pathogen collected in the collection (S5a). Specifically, the detector 13 performs a pretreatment, causes the reaction in the reactor 13a to be performed for the second time period shorter than the first time period in the second detection mode as illustrated in FIG. 12, and detects the pathogen on the basis of the intensity of fluorescence when the second time period elapses from the start of detection (i.e., the start of reaction). At this time, the detector 13 may detect the quantity of labeled substance reacted with the pathogen.

In a case where the body temperature of the subject obtained by the obtainer 11 is higher than the first threshold value (YES in S2), the controller 14 sets the second control mode (S7a). Accordingly, the controller 14 controls the collector 12 in the second collection mode and controls the detector 13 in the second detection mode without a pretreatment, as illustrated in FIG. 18.

Subsequently, in the second control mode, the collector 12 collects a pathogen carried by the subject or a pathogen in the air around the subject (S8a). Specifically, the collector 12 collects a pathogen for the fourth time period shorter than the third time period in the second collection mode, as described above with reference to FIG. 14.

Subsequently, in the second control mode, the detector 13 detects the pathogen collected in the collection (S9a). Specifically, the detector 13 omits a pretreatment, causes the reaction in the reactor 13a to be performed for the second time period shorter than the first time period in the second detection mode as illustrated in FIG. 12, and detects the pathogen on the basis of the intensity of fluorescence when the second time period elapses from the start of detection (i.e., the start of reaction). At this time, the detector 13 may detect the quantity of labeled substance reacted with the pathogen.

The reporter 15 reports a detection result obtained by the detector 13 in step S5a, step S9a, or step S13 (S6a).

### Fifth Modification Example

In the above-described embodiment, the obtainer 11 obtains a body temperature of a subject by measuring the body temperature of the subject. The embodiment is not limited thereto, and the obtainer 11 may obtain a body temperature of a subject by receiving an input of a result obtained by measuring the body temperature of the subject using a thermometer or the like.

### Sixth Modification Example

In the above-described embodiment, the pathogen detection apparatus 10 is configured to collect breath exhaled by a subject directly from the subject. The embodiment is not limited thereto, and the pathogen detection apparatus 10 may be installed in a room where people come in and out and may be configured to collect the air in the space of the room.

In the pathogen detection apparatus 10 having this configuration, the obtainer 11 obtains a body temperature of one or more subjects present in the space of the room. In a case where the body temperature of any one of the one or more subjects is higher than a predetermined threshold value, the controller 14 controls at least one of the collector 12 or the detector 13 to shorten the time period from the start of collection by the collector 12 to the report of a detection result by the reporter 15.

In the above-described embodiment, the individual components may be constituted by dedicated hardware or may be implemented by executing a software program suitable for the individual components. The individual components may be implemented when a program executing unit of a CPU or processor reads out and executes the software program recorded on a recording medium, such as a hard disk or a semiconductor memory. Here, the software that implements the pathogen detection apparatus and the pathogen detection method according to the above-described embodiment is the following program.

The program causes a computer to execute a pathogen detection method including: obtaining a body temperature of a subject; determining a control mode in accordance with the obtained body temperature of the subject; collecting, in the determined control mode, a pathogen carried by the subject or a pathogen in air around the subject; detecting, in the determined control mode, the pathogen collected in the collecting; and reporting a detection result obtained in the detecting. In the determining the control mode, (1) the control mode is determined to be a first control mode in a case where the obtained body temperature of the subject is lower than or equal to a predetermined threshold value, and (2) the control mode is determined to be a second control mode in a case where the obtained body temperature of the subject is higher than the predetermined threshold value, a time period from start of collection of the pathogen to report of the detection result being shorter in the second control mode than in the first control mode.

The pathogen detection apparatus and the pathogen detection method according to an aspect or aspects of the present disclosure have been described on the basis of the embodiment. The present disclosure is not limited to the embodiment. An embodiment established by applying a modification conceived by a person skilled in the art to the above embodiment, and an embodiment established by combining components in different embodiments may be included in the scope of an aspect or aspects of the present disclosure without deviating from the gist of the present disclosure.

The present disclosure is useful as a pathogen detection apparatus and a pathogen detection method that are capable of efficiently detecting a pathogen from a subject or a space around the subject.

What is claimed is:

1. A pathogen detection apparatus comprising:
an obtainer that obtains a body temperature of a subject;
a collector that collects a pathogen carried by the subject or a pathogen in air around the subject;
a detector that performs detection of the pathogen collected by the collector;
a reporter that reports a detection result obtained by the detector; and
a controller, wherein
in a case where the body temperature of the subject obtained by the obtainer is higher than a predetermined threshold value, the controller controls at least one of the collector or the detector to shorten a time period from start of collection by the collector to report of the detection result by the reporter.

2. The pathogen detection apparatus according to claim 1, wherein
in a case where the body temperature of the subject obtained by the obtainer is lower than or equal to the predetermined threshold value, the controller controls the detector in a first detection mode in which the detector detects the pathogen for a first time period, and
in a case where the body temperature of the subject is higher than the predetermined threshold value, the controller controls the detector in a second detection mode in which the detector detects the pathogen for a second time period shorter than the first time period.

3. The pathogen detection apparatus according to claim 2, wherein
the detector includes
a reactor that causes a reaction to occur between the pathogen collected by the collector and a labeled substance, and
a light irradiator that irradiates, with excitation light, a reacted substance obtained through the reaction in the reactor,
in the first detection mode, the detector detects the pathogen on the basis of fluorescence generated by the labeled substance as a result of irradiating, with the excitation light, the reacted substance obtained through the reaction for the first time period, and
in the second detection mode, the detector detects the pathogen on the basis of the fluorescence generated by the labeled substance as a result of irradiating, with the excitation light, the reacted substance obtained through the reaction for the second time period shorter than the first time period.

4. The pathogen detection apparatus according to claim 1, wherein
the detector is capable of performing, on the pathogen collected by the collector, a pretreatment for promoting the detection,
in a case where the body temperature of the subject obtained by the obtainer is lower than or equal to the predetermined threshold value, the controller causes the detector to perform the pretreatment in the detection, and
in a case where the body temperature of the subject is higher than the predetermined threshold value, the controller causes the detector to omit the pretreatment in the detection.

5. The pathogen detection apparatus according to claim 1, wherein
in a case where the body temperature of the subject obtained by the obtainer is lower than or equal to the predetermined threshold value, the controller controls the collector in a first collection mode in which the collector collects the pathogen for a third time period, and
in a case where the body temperature of the subject is higher than the predetermined threshold value, the controller controls the collector in a second collection mode in which the collector collects the pathogen for a fourth time period shorter than the third time period.

6. A pathogen detection method comprising:
obtaining a body temperature of a subject;
determining a control mode in accordance with the obtained body temperature of the subject;
collecting, in the determined control mode, a pathogen carried by the subject or a pathogen in air around the subject;
detecting, in the determined control mode, the pathogen collected in the collecting; and
reporting a detection result obtained in the detecting, wherein
in the determining the control mode, (1) the control mode is determined to be a first control mode in a case where the obtained body temperature of the subject is lower than or equal to a predetermined threshold value, and (2) the control mode is determined to be a second control mode in a case where the obtained body temperature of the subject is higher than the predetermined threshold value, a time period from start of collection of the pathogen to report of the detection result being shorter in the second control mode than in the first control mode.

7. A pathogen detection method comprising:
obtaining a body temperature of a subject;
collecting air around the subject;
loading a liquid generated from the air to an irradiation target portion that has first antibodies disposed on a monolayer disposed on a metal layer, the liquid containing pathogens contained in the air, second antibodies that bind to the pathogens, and labeled substances that bind to the second antibodies;
outputting information that is based on an intensity of fluorescence generated by irradiating, for a predetermined time period, with excitation light, the irradiation target portion to which the generated liquid has been loaded, the fluorescence being reflection light reflected by the labeled substances; and
determining a concentration of the pathogens on the basis of the output information, wherein
in a case where the body temperature is lower than or equal to a predetermined value, the predetermined time period is a first time period,
in a case where the body temperature is higher than the predetermined value, the predetermined time period is a second time period shorter than the first time period, and
when irradiated with the excitation light, the metal layer causes an intensity of fluorescence generated by a labeled substance that binds to an antibody included in the first antibodies among the labeled substances to be higher than an intensity of fluorescence generated by a labeled substance that does not bind to an antibody included in the first antibodies among the labeled substances.
